(19) United States
(12) Patent Application Publication
(10) Pub. No.: US 2003/0078660 A1
(43) Pub. Date: Apr. 24, 2003

(54) Orthopedic implant and method for orthopedic treatment

(76) Inventors: Dale Clifford, Portland, OR (US); Mark Manasas, Dedham, MA (US)

Correspondence Address: DREIER & BARITZ LLP, 499 PARK AVENUE, 20TH FLOOR, NEW YORK, NY, 10022, US

(21) Appl. No.: 10/076,122

(22) Filed: Feb. 14, 2002

Related U.S. Application Data
(60) Provisional application No. 60/269,074, filed on Feb. 15, 2001.

(51) Int. Cl. A61F 2/44; A61F 2/28

(52) U.S. Cl. 623/17.11; 623/17.16; 623/23.54

(57) ABSTRACT

The present invention is directed to an orthopedic implant that is comprised of a corrugated foraminous sleeve. The sleeve is formed with alternating grooves and ridges, referred to herein as lobes and depressions. The corrugated sleeve may be formed from a sheet provided with openings (foramina), which is then corrugated to impart the lobes and depressions. The sheet may then be enclosed in a loop, such as a circular shape, elliptical shape, or any other shape contemplated by the skilled artisan, to form a corrugated cage that may be used as an orthopedic implant.

In another embodiment of the invention, the corrugated foraminous implant is formed from a pre-formed foraminous loop, such as a loop having a circular shape, an elliptical shape, or any other shape contemplated by a skilled artisan. The loop is processed to impart the corrugated nature of the invention, as manifested in the lobes and depressions of the implant. The preformed loop can be an endless loop having no discernible point where two ends are joined.

### CROSS-REFERENCE TO RELATED APPLICATIONS

This application claims the benefit under 35 U.S.C. 119(e) of U.S. Provisional Application Serial No. 60/269,074, filed Feb. 15, 2001.

### FIELD OF THE INVENTION

The present invention is directed to an orthopedic implant, a method of providing the orthopedic implant and a method for orthopedic treatment using the orthopedic implant.

### BACKGROUND OF THE INVENTION

Irregularities and abnormalities affecting the intervertebral discs are often the cause of acute, subacute and chronic back pain. These discs provide spacing, articulation, and cushioning between the vertebrae. If the normal properties of the discs are compromised, performance of these functions can be adversely affected. Disc collapse or narrowing may reduce the space between vertebrae, and damage to the disc can cause it to bulge or rupture, possibly extruding into the spinal canal or neural foramen. These changes can cause debilitating back and distal pain, numbness, or weakness.

Severe cases can be surgically treated by microsurgical laminectomy, or minimally invasive surgical techniques to remove central or extruded disc material, or by removal of the entire damaged disc and fusion of the adjacent vertebrae. The approach to lumbar fusion can either be anterior or posterior. Anterior fusion techniques employ Interbody Fusion Devices (IBFD's). These devices are inserted from an anterior approach into the space vacated by the disc to cause space retention, stabilization and load bearing. Posterior fusion is accomplished by cutting through the musculature of the back, exposing the spinal segments, and using the appropriate components, such as metal rods, screws, and other devices. Also, bone may be packed into the intervertebral space to induce fusion. In packing bone, Autograft techniques may be employed in which bone harvested from the patient's iliac crest. Allograft techniques may also be employed, where bone is taken from donor. Also, synthetic biocompatible material may be packed into the space to induce fusion.

Other kinds of orthopedic injuries present issues relating to repair. Broken bones are conventionally treated by setting the bone and using a cast, brace, or similar external support to hold the bone together to allow healing. This type of treatment is most suited to the repair of simple breaks in long bones, such as the femur. For other bones, or fractures involving several bone fragments, various devices, such as pins, rods, surgical mesh and screws have been used to join the bone in the proper orientation for repair. These techniques have the disadvantage of being invasive to the bone itself, possibly resulting in further weakening of the damaged tissue.

### SUMMARY OF THE INVENTION

The present invention is directed to an orthopedic implant that is comprised of a corrugated foraminous sleeve. The sleeve is formed with alternating grooves and ridges, referred to herein as lobes and depressions. The corrugated sleeve may be formed from a sheet provided with openings (foramina), which is then corrugated to impart the lobes and depressions. The sheet may then be enclosed in a loop, such as a circular shape, elliptical shape, or any other shape contemplated by the skilled artisan, to form a corrugated cage that may be used as an orthopedic implant.

In another embodiment of the invention, the corrugated foraminous implant is formed from a pre-formed foraminous loop, such as a loop having a circular shape, an elliptical shape, or any other shape contemplated by the skilled artisan. The loop is processed to impart the corrugated nature of the invention, as manifested in the lobes and depressions of the implant. The preformed loop can be an endless loop having no discernible point where two ends are joined.

The orthopedic implants of the present invention are constructed of a biocompatible material, such as titanium. The foraminous nature of the implant is manifested in the apertures or openings (foramina) that are dispersed among the landed regions, which have the appearance of a lattice structure.

The landed regions form an intersecting network, which surround the openings in the implant. The intersecting network of landed regions provides the structural support for the implant, and permits it to assume load bearing responsibility when, for example, the implant is inserted in the space between two vertebrae. Preferably, the implant is made of titanium, a strong material that is biocompatible. Other materials may be used, such as cobalt chromium.

The openings in the implant permit the free flow of materials, such as bodily fluids, into and out of the implant. Also, where the implant is employed to promote spinal fusion, bone is capable of growing through the openings in the implant. Particularly, this is the case where the implant, prior to implantation, has been packed with bony material. When implanted successfully, fusion should occur at sites inside and outside of the implant, due to bone growth through the openings in the implant.

The corrugations in the implant are manifested in an alternating arrangement of lobes and depressions that are formed in the material. It is believed that the corrugated structure of the implant enhances the strength of the implant. That is, if two implants were formed of the same material, had the same size openings, had the same pattern of openings, as well as the same height, width, and sheet thickness, with the only difference in the implants being that one is corrugated and the other is not corrugated, then the corrugated implant will exhibit better strength properties than the noncorrugated implant. More particularly, the corrugated implant will exhibit load bearing properties superior to those of the noncorrugated implant. The implant will exhibit greater resistance to compression, bucking and bending. In a practical sense, this means that the sheet thickness of a corrugated implant can be thinner than the sheet thickness of a noncorrugated implant, while exhibiting substantially the same strength properties. This may provide an additional volume in which bone material can be packed into the implant. Alternatively, the skilled artisan may be able to employ a corrugated implant bearing a greater degree of openness than a non-corrugated implant, which facilitates bone growth through the openings of the implant.

The applicant has found that the implants may be provided with four lobes, six lobes, or any other number of lobes that is desired. The applicant has found that four lobes are well suited for relatively smaller sized implants, such as smaller, circular shaped implants. On the other hand, six lobes are well-suited larger sized perimeters, such as where the implant has an elliptical shape.

The “sheets” used in the present invention, or the “loops”, as the case may be, are relatively thin materials. That is, the walls of the implant have a thickness dimension in the size range of about 0.5 mm to about 3.0 mm.

In another embodiment, the corrugated foraminous structure is employed as the inner sleeve of a structure in which a second sleeve is sized to surround the inner sleeve. The outer sleeve is foraminous, and formed of a biocompatible material.

Another embodiment of the present invention includes a method of providing an orthopedic implant, including the steps of providing a sheet suitable for construction into a corrugated implant, designing a shape, size and position of openings to be made in the sheet to provide a lattice that is a network of intersecting landed regions, and forming the orthopedic implant.

Another embodiment of the present invention includes a method of providing an orthopedic implant, including the steps of providing a loop having a selected size and shape, the loop being suitable for construction into a corrugated implant, and having a selected shape, size and position of openings in the sheet which defines a lattice that is a network of intersecting landed regions, and forming the orthopedic implant.

According to another embodiment of the invention, a method of orthopedic treatment is provided, including encircling an area of a bone with a sheet to form a corrugated and foraminous sleeve having a longitudinal axis, and securing the sleeve around the bone.

In yet another embodiment of the invention, a method is provided in which the aforedescribed implants are implanted in the disc space between adjacent vertebrae, or alternatively, in the space where adjacent discs and vertebrae have been removed from the spinal column.

### BRIEF DESCRIPTION OF THE DRAWINGS

FIG. 1a is a side elevational view of one embodiment of an orthopedic implant of the invention;

FIG. 1b is a cross-sectional view of the embodiment of the orthopedic implant shown in FIG. 1a;

FIG. 2a is a side elevational view of another embodiment of an orthopedic implant of the invention;

FIG. 2b is a cross-sectional view of the embodiment of the orthopedic implant shown in FIG. 2a;

FIG. 3a is a side elevational view of another embodiment of an orthopedic implant of the invention;

FIG. 3b is a cross-sectional view of the embodiment of the orthopedic implant shown in FIG. 3a;

FIG. 4 is a perspective view of another embodiment of an orthopedic implant of the invention;

FIG. 5a is a side elevational view of another embodiment of an orthopedic implant of the invention;

FIG. 5b is a cross-sectional view of the embodiment of the orthopedic implant shown in FIG. 5a;

FIG. 5c is a perspective view of the embodiment of the orthopedic implant shown in FIG. 5a;

FIG. 6 is a perspective view of another embodiment of an orthopedic implant of the invention;

FIG. 7 is a lateral view of another embodiment of an orthopedic implant of the invention;

FIG. 8 is a transverse view of the embodiment of the invention shown in FIG. 7;

FIG. 9 is a perspective view of another embodiment of an orthopedic implant of the invention;

FIG. 10 is a cross-sectional view of another embodiment of an orthopedic implant of the invention;

FIG. 11 a is a side elevational view of another embodiment of an orthopedic implant of the invention;

FIG. 11b is a cross-sectional view of the embodiment of the invention shown in FIG. 11a;

FIG. 12 is a perspective view of an embodiment of the present invention;

FIG. 13 is a cross sectional view of the embodiment shown in FIG. 12;

FIG. 14 is a diagram showing, in cross section, the dimensional transformation of the FIG. 12 embodiment;

FIG. 15 is a perspective view of an embodiment of the present invention;

FIG. 16 is a cross sectional view of the embodiment shown in FIG. 15; and

FIG. 17 is a diagram showing, in cross section, the dimensional transformation of the FIG. 15 embodiment.

### DETAILED DESCRIPTION OF THE PREFERRED EMBODIMENTS

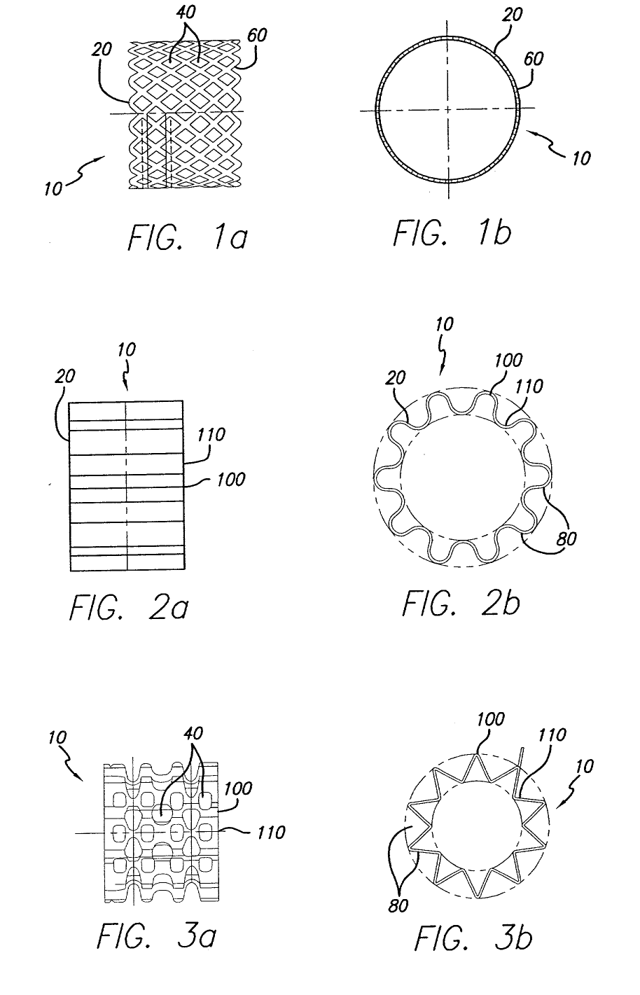

FIGS. 1a and 1b show a foraminous loop 10′ in the shape of a circle. Loop 10′ comprises a sleeve 20 constructed from a sufficiently strong, biocompatible material. The sleeve 20 includes openings 40 that are interspersed among the landed regions 60. Here, the landed regions intersect each other to form diamond shaped openings, but it should be readily understood that other openings shapes, such as squares, circles, rectangles, and others are possible. FIGS. 2a and 2b show sleeve 20 after it has been processed in order to impart corrugations 80.

The embodiment of implant 10 illustrated in FIGS. 1a and 1b includes openings 40 sized, shaped and positioned to impart the desired degree of implant strength and openness. The network of landed regions create a supporting lattice 60. This embodiment may be placed around a bone to provide support during treatment, or it may be used as a spacer in the spinal column. That is, it can be inserted in the disc space between adjacent vertebrae, where it serves as a load-bearing component.

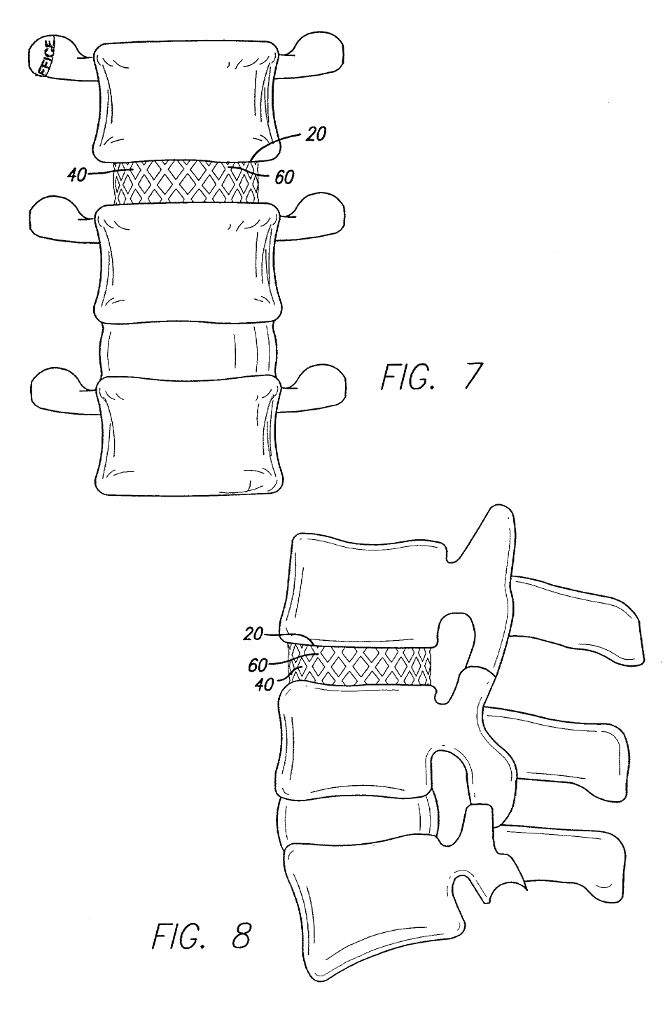

When placed between vertebrae, implant 10 can be inserted lengthwise in the gap in the spine between two vertebrae 210, as illustrated in FIGS. 7 and 8. For this application, the openings and corrugations 60 may be sized, shaped and positioned to insure load-bearing properties that support the spine. When bony material is packed inside the implant, the openings in the implant promote bone fusion between the vertebrae. Alternatively, the implant can be employed as a replacement for adjacent discs and at least one vertebral body that have been removed from the spinal column. For example, the implant can replace a vertebra and the discs positioned above and below the vertebra, thereby providing support for the spinal column in lieu of the removed discs and vertebra. In this arrangement, the implant will be employed with a pedicle screw system that employs pedicles screws, receiver members, and elongated rods, such as the system disclosed in U.S. patent application Ser. No. 09/749,099 filed Dec. 18, 2000, itself a continuation of U.S. patent application Ser. No. 09/407,044 filed Sep. 27, 1999. These applications are incorporated by reference herein.

As a support for a bone, implant 10 may be placed around a bone during treatment. For this application, openings 40 may be sized, shaped and positioned to support the bone along its length to prevent displacement, while allowing the bone to support itself during treatment and receiving adequate access to nutrients.

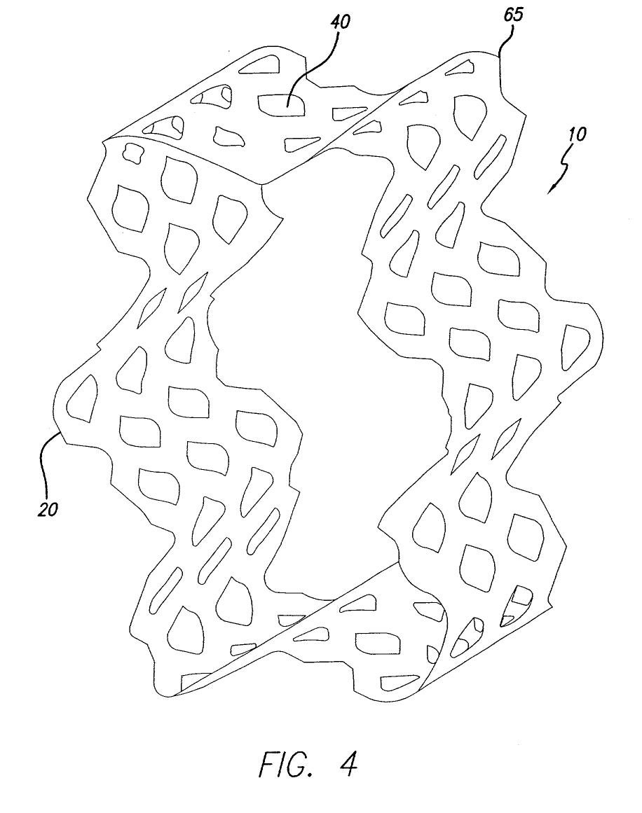

The embodiment of implant 10 illustrated in FIGS. 2a and 2b includes corrugations 80. As used herein, the “corrugation” refers to series of bends or folds creating waves or undulations having a series of peaks 100 and troughs 110. Corrugations 80 may follow any function or pattern. For example, corrugations 80 could form an “S” curve as illustrated in FIGS. 2a and 2b, a triangle pattern, as illustrated in FIGS. 3a and 3b, or other patterns such as a saw tooth pattern, or a square wave pattern, or the lobes and depressions of FIGS. 4, 12, 13, 15 and 16.

As shown in FIG. 6, implant 10 can be placed around a bone 200 to provide support along the length of bone 200 during treatment of the bone. Alternatively, implant 10 having corrugations 80 can be placed in a gap in the spine, such as between two vertebrae 210, as a spacer element, as shown in FIGS. 7 and 8. It is believed that because the foraminous implant is corrugated, the wall thickness of the implant can be lesser than an implant with a thicker sleeve that is not corrugated.

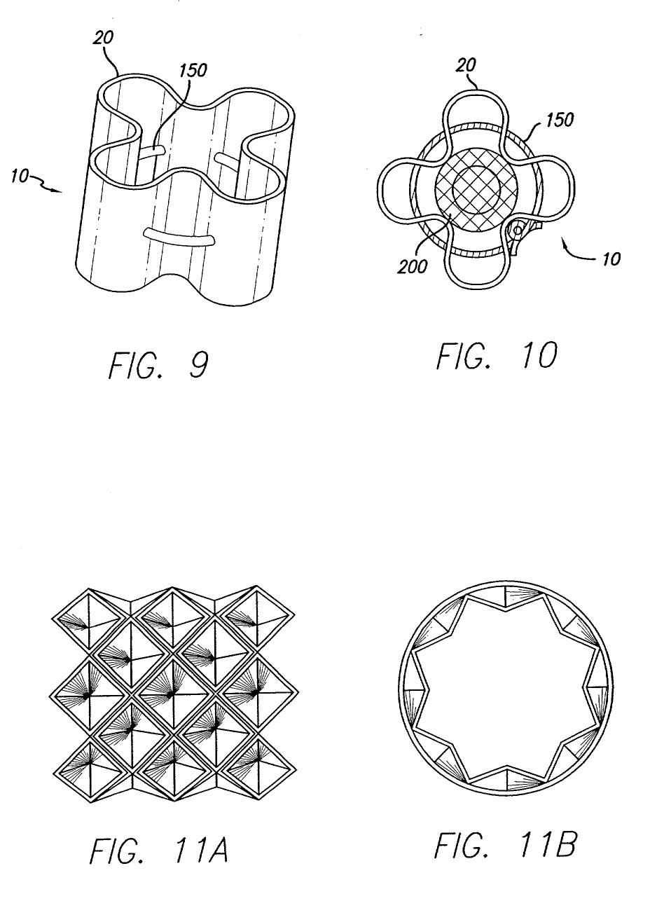

Implants 10 may also be installed with a cerclage 150. As used herein, a cerclage is a piece of material which encircles a sleeve, and holds the sleeve together and/or fixes the sleeve in place around a bone. As illustrated in FIGS. 9 and 10, cerdlage 150 may be threaded through perforations 40 and passed through corrugations 80, and fixing sleeve 20 in place.

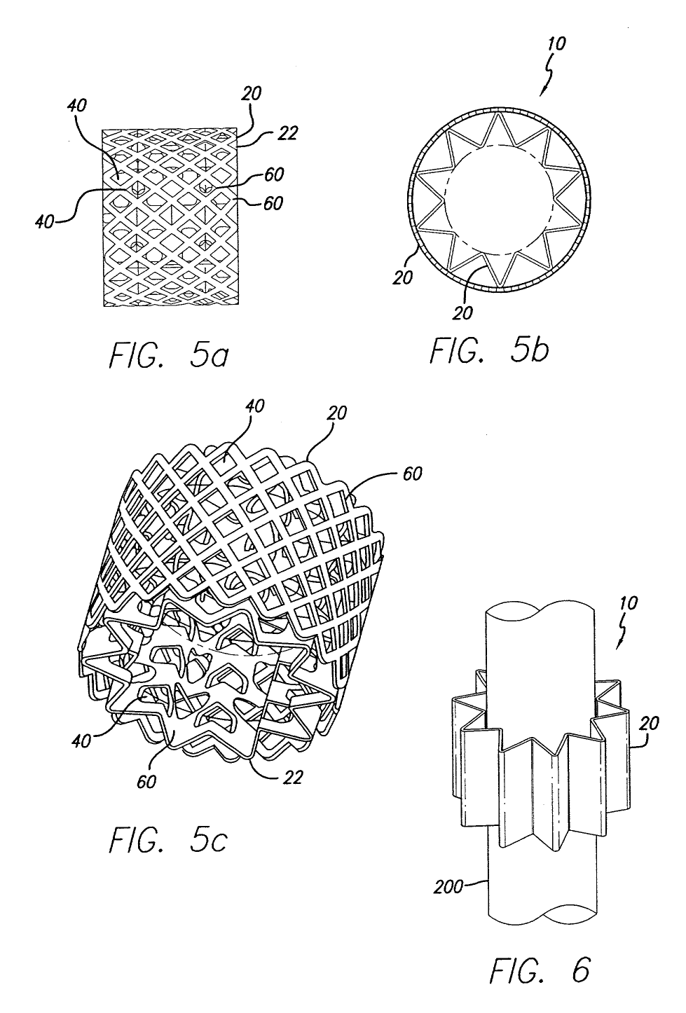

Other embodiments of implant 10 may have multiple, concentric sleeves 20, 22. FIGS. 5a, 5b and 5c illustrate an embodiment in which an internal sleeve 22 having perforations 40 and corrugations 80 is surrounded by an outer sleeve 20 having perforations 40.

Sleeve 20 of implant 10 may have geometry other than corrugations 80 and perforations 40. For example, as illustrated in FIGS. 11a and 11b, a sleeve 20 may have a series of indentations or protrusions 160 sized, shaped and positioned to tune any of the physical properties of the implant.

Any embodiment of implant 10 may include bone graft or bone growth material within sleeve 20. In bone repair applications this material may be used to promote healing or to replace an area of bone that is missing or damaged.

One embodiment of the invention is a method of providing an orthopedic implant, the method comprised of: providing a first sheet suitable for construction into an implant 10, designing the shape, size and position of openings 40 to be made providing a supporting lattice 60 of intersecting landed regions and forming the sheet into an implant 10. The ability to create the implant 10 from a sheet is one way to make the invention. This method allows the sleeve to be non-invasively installed around a bone 200. In another method, the loop is preformed and the implant is designed by selecting the size, shape and position of the openings 10 and the corrugations 80. The corrugations are then imparted to the loop, thereby forming the implant.

Any embodiment of the method of providing an orthopedic implant may further include providing a second sheet constructed into an outer sleeve 20, and adapted to surround inner sleeve 22, and forming the outer sleeve 20 around inner sleeve 20 to construct implant 10.

The present invention further includes a method of orthopedic treatment in which the corrugated implant is installed as a spacer element, in the disc space between adjacent vertebrae. That is, a sheet is formed into a sleeve 20, the sheet having perforations 40 and a corresponding supporting lattice 60 of landed regions. The sleeve is implanted into the space between two vertebrae.

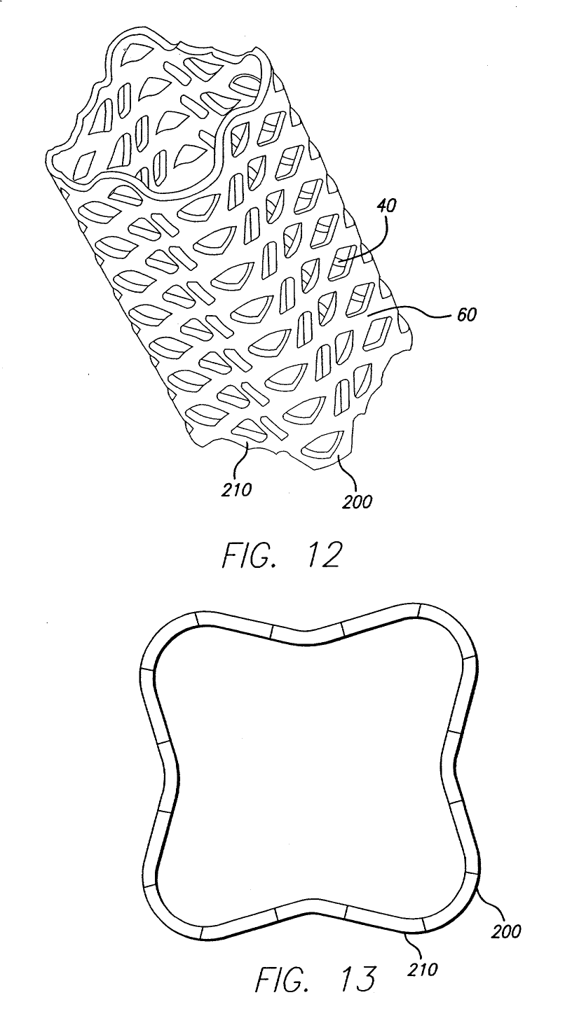

FIG. 12 shows a, corrugated implant in a perspective view. FIG. 13 shows the same implant shown in cross section. The implant is provided with diamond shaped openings 40 interspersed between the landed regions of biocompatible material, which can be titanium, due to its excellent strength properties, relatively light weight and biocompatibility. While the skilled artisan would understand that other suitable materials are available, titanium is recognized well suited because it delivers a combination of desirable properties.

As seen in FIGS. 12 and 13, the corrugated implant is formed of a material whose thickness dimension is sheet like. That is, the size thickness dimension is far less than the sizes of the height and width dimension, as is the case with sheet-like materials. Here as shown in FIGS. 12 and 13, the implant is actually formed out of an endless loop of material which has the sheet-like properties described herein.

The “sheets” used in the present invention, or the “loops”, as the case may be, are relatively thin materials. That is, the walls of the implant have a thickness dimension in the size range of about 0.5 mm to about 3.0 mm.

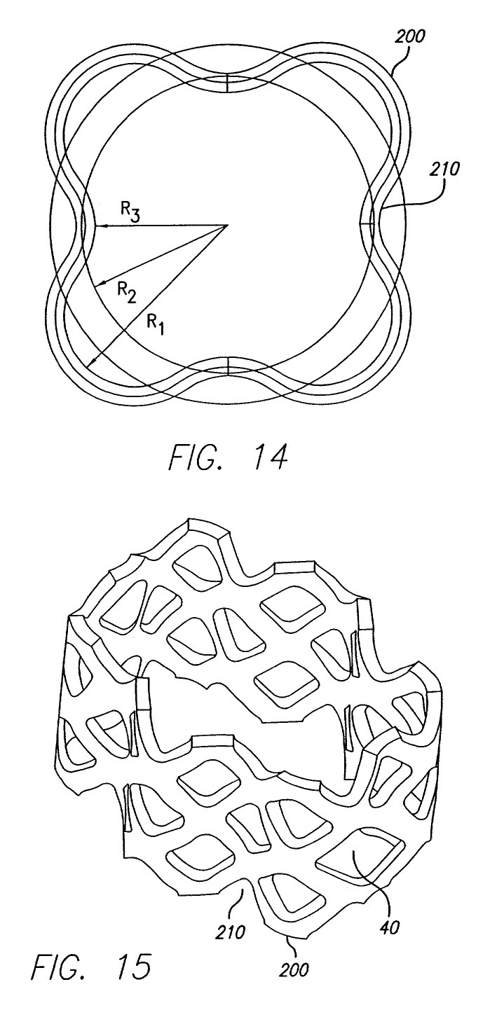

FIGS. 12 and 13 show a corrugated structure characterized by four lobes, designated 200, and four depressions, designated 210. FIG. 14 shows the cross section of the implant, after it has been corrugated, interposed over the cross-section of the loop prior to the corrugation process (which in this case is a circular cross section). As shown in this Figure the radius R₁ of the lobe 200, as measured to the inner wall of the lobe, is greater than the radius R₂ of the loop, prior to corrugation. On the other hand, the radius R₃ of the depression 210 as measured to the inner wall of the depression, is less than the radius of the loop R₂. While FIG. 14 facilitates an understanding of one aspect of the invention, the term “corrugated”, as used herein, is intended to have its ordinary meaning, that is, a thing shaped into alternating grooves and ridges.

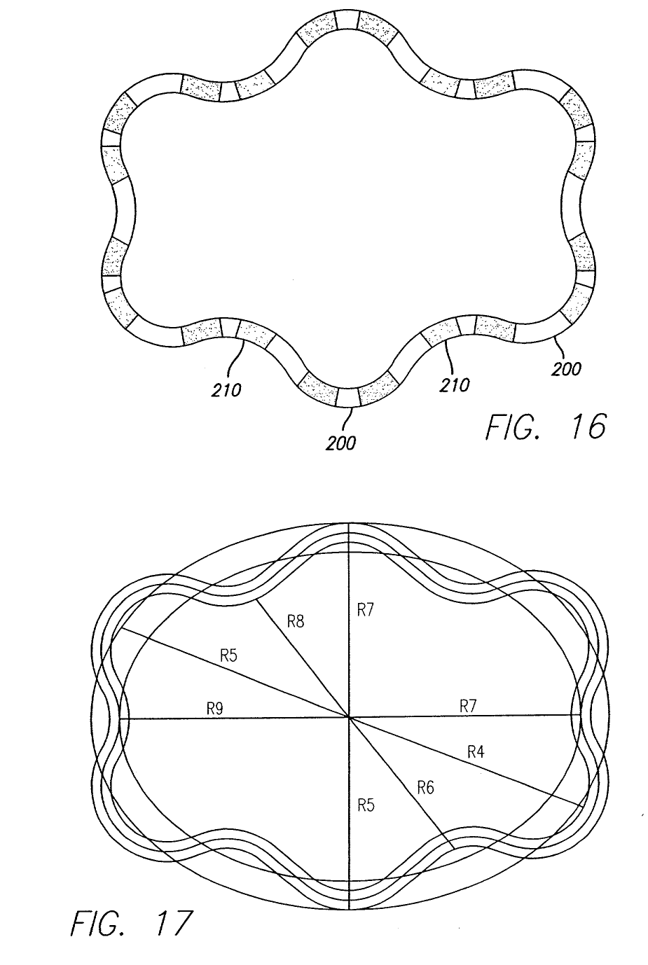

FIGS. 15 and 16 show a corrugated implant that is similar to the implants of FIGS. 12 and 13, with the exception being the implant of FIGS. 15 and 16 has six lobes 200 and six depressions 210. The embodiment of FIGS. 15 and 16, prior to corrugation, is formed from an elliptically shaped loop. FIG. 17 shows the cross section of the implant, after it has been corrugated, interposed over the cross-section of the elliptical loop, prior to corrugation. As shown in this Figure, the radii R₄, R₅ of the lobe 200, as measured to the inner wall of the lobe, is greater than the radii R₆, R₇ of the loop taken at the respective locations prior to formation of the corrugations. Likewise, the radii R₈, R₉ of the depressions, as measured to the inner wall of the depression, is less than the radii R₆, R₇ of the loop taken at the respective locations prior to formation of the corrugations. While FIG. 17 facilitates an understanding of one aspect of the invention, the term “corrugated”, as used herein, is intended to have its ordinary meaning, that is, a thing shaped into alternating grooves and ridges.

The implant of the present invention can be formed in a variety of ways, as would readily be appreciated by the person skilled in the art. For example, where a tube or loop is used to form the implant, laser cutting can be employed to cut the openings in the implant. The corrugations can be formed by placing the loop or tube over a mandrel having the finished shape of the implant, and applying sufficient pressure on all sides to form the lobes and depressions.

Where a sheet is employed, the sheet can be bent in order to form the loop, which can then be welded together. The corrugations can be imparted by employing a mandrel and applying pressure, as discussed above.

In addition to laser cutting, stamping, or chemically etching techniques can be employed to create the openings in the implant.

Having thus described at least one preferred embodiment of the implant and method of the invention, various alterations, modifications and improvements will readily occur to those skilled in the art. Such alternations, modifications and improvements are intended to be part of the disclosure and to be within the spirit and scope of the invention. Accordingly, the foregoing description is by way of example only and is limited only as defined in the following claims and equivalents thereto.

We claim:

1. An orthopedic implant, comprising: a foraminous, corrugated biocompatible material formed into a sleeve.

2. The orthopedic implant of claim 1, wherein the orthopedic implant is provided with a first and second end and a length dimension extending therebetween, wherein the first and second ends are open.

3. The orthopedic implant of claim 1 wherein the implant is provided with a plurality of lobes and depressions.

4. The orthopedic implant of claim 1 wherein the biocompatible material is titanium.

5. The orthopedic implant of claim 1 wherein the walls of the implant have a thickness dimension in the size range of about 0.5 mm to about 3.0 mm

6. The orthopedic implant of claim 1 wherein the implant is provided with four lobes and four depressions.

7. The orthopedic implant of claim 1 wherein the implant is provided with six lobes and six depressions.

8. The orthopedic implant of claim 1 wherein the implant is constructed from a foraminous corrugated loop.

9. The orthopedic implant of claim 1 wherein the implant is constructed from a foraminous corrugated sheet.

10. The orthopedic implant of claim 1 wherein the implant is comprised of an intersecting network of landed regions that define a plurality of openings in the network, wherein the openings are dispersed among the landed regions.

11. The orthopedic implant of claim 1 wherein the implant has a substantially circular shape.

12. The orthopedic implant of claim 1 wherein the implant has a substantially elliptical shape.

13. The orthopedic implant of claim 1, wherein the implant surrounds a material selected from the group consisting of bone graft material and a bone growth promoting material and mixtures thereof.

14. The orthopedic implant of claim 1, further comprising a cerclage passing through the openings and secured around the sleeve.

15. The orthopedic implant of claim 1, wherein the orthopedic implant occupies the disc space between two vertebrae.

16. The orthopedic implant of claim 1, wherein the sleeve is an inner sleeve and the implant further comprises an outer sleeve adapted to surround the inner sleeve.

17. A method of providing an orthopedic implant, comprising:
providing a sheet suitable for construction into a sleeve;
selecting the shape, size and position of openings and corrugations to be made in the sheet;
selecting a biocompatible material;
forming the sheet according to the design; and
enclosing the sheet to form the implant.

18. The method of claim 17, further comprising:
encircling an area of a bone with a formed sheet to form a sleeve having openings and corrugations; and
securing the sheet around the bone.

19. Method of claim 18, wherein the step of securing the sheet around the bone further comprises threading a cerclage through the perforations and corrugations and affixing the ends of the cerclage.

20. [Intentionally Left Blank]

21. [Intentionally Left Blank]

22. A method of orthopedic treatment, comprising implanting the implant of claim 1 into the space between two vertebrae.

23. The method of claim 22 wherein bone is placed in the implant prior to implanting.

24. A method of providing an orthopedic implant, comprising:
Providing a loop suitable for construction into a sleeve;
Selecting the shape, size and position of openings and corrugations to be made in the sheet;
selecting a biocompatible material; and
forming the implant according to the design.
